(19) United States
(12) Patent Application Publication
(10) Pub. No.: US 2011/0021958 A1
(43) Pub. Date: Jan. 27, 2011

(54) Therapeutic Devices And Methods Of Using The Same

(76) Inventor: Bruce G. Lynds, Hudsonville, MI (US)

Correspondence Address: COOK ALEX LTD, SUITE 2850, 200 WEST ADAMS STREET, CHICAGO, IL, 60606, US

(21) Appl. No.: 12/509,961

(22) Filed: Jul. 27, 2009

(51) Int. Cl. A61H 1/00; A61B 17/00

(52) U.S. Cl. 601/34; 606/201

(57) ABSTRACT

Therapeutic devices and methods for reducing the incidence and severity of symptoms related to abnormal neurologic activity within a body part are disclosed. The therapeutic device includes at least one holding implement configured to position the therapeutic device on the body part and at least one therapeutic implement connected to the holding implement and configured to provide light tactile stimulation to a region of the body part that is in the vicinity of the holding implement.

### FIELD OF THE DISCLOSURE

The present disclosure generally relates to therapeutic devices and methods for controlling muscle function and/or relieving neuropathic discomfort, and more particularly for treating neurological conditions or disorders that involve abnormal neurologic activity.

### BACKGROUND

Certain neurological conditions or disorders involve inappropriate muscle activity or control, idiopathic peripheral parathesia (abnormal sensations having an unknown cause), or dysesthesias (unpleasant abnormal sensations), collectively “abnormal neurologic activity” in body parts. An example of such disorders is Restless Legs Syndrome (RLS), which can be a life-long condition for which there is no known cure. Individuals affected by the disorder may describe the symptoms as pulling, crawling, tingling, prickly, or painful sensations that are usually accompanied by a strong urge to move the legs. Patients having a further example of such disorders, known as Periodic Limb Movement Disorder (PLMD), may experience sudden muscle jerks or involuntary movements, typically in the arms or legs. A patient may have RLS and not PLMD, or vice versa, and a patient may be afflicted with both disorders. In turn, both disorders may cause interrupted sleep.

The term “body part” is used herein to more broadly refer to any portion of an individual's body, whether it be a peripheral limb or appendage, peripheral surface of the body, or other portion of the trunk of a person. The disorders may affect various body parts, such as with PLMD, where the arms also may be subject to symptoms. Nevertheless, the present disclosure provides therapeutic devices and methods that treat these as well as other disorders that involve abnormal neurologic activity, such as the aforementioned inappropriate muscle activity, idiopathic peripheral parathesia or neuropathic discomfort.

Returning to the example of RLS, various methods for treating symptoms associated with RLS are known in the art. Many methods involve administering drugs of various types. For example, U.S. Pat. No. 6,855,735 discloses administering a dose of ketamine to alleviate symptoms associated with RLS. Other examples of treatments involving the use of drugs include U.S. Pat. No. 6,716,854, which discloses the use of heterocyclic amines, and phenylazacycloalkane compounds for the treatment of RLS, and U.S. Pat. No. 6,544,565, which discloses the use of Valeriana. Each of the many various treatments involving administering drugs requires at least the expenses of visiting a doctor, ongoing purchases of the medications, attention to potential adverse drug interactions with other medications the patient may be taking, and the possibility of experiencing side effects from the RLS medication that may or may not be known or recognized.

Other methods for treating symptoms associated with RLS or disorders involving inappropriate muscle movements include applying pressure or applying pressure intermittently to an extremity of the patient, such as is disclosed by Morgenlander in U.S. Patent Application Publications 20030176822 and 20050026912, fitting an apparatus to a leg of a person to restrain the movement of the foot as disclosed by Wilcocks in U.S. Patent Application Publication 20080039303, and the application of electrical stimulation, as is disclosed by Karrell in U.S. Pat. No. 5,759,198

### SUMMARY

The present disclosure is directed to therapeutic devices and methods of using the devices for treating and relieving symptoms related to disorders that include abnormal neurologic activity, as will be set forth in or otherwise apparent from the description and drawings that follow, as well as will be learned by the practice of the subject matter disclosed herein.

The discovery that application of a passive device to provide light tactile stimulation, as disclosed herein, can provide a remarkable reduction in or the elimination of the symptoms related to RLS and PLMD is both surprising and unanticipated. While the devices and methods disclosed have proven to be effective in reducing the incidence and severity of symptoms related to RLS and PLMD, the devices and methods may be used to reduce the incidence and severity of unexpected limb movements or abnormal sensations experienced by patients afflicted with Multiple Sclerosis (MS), or the incidence and severity of pain caused by dysfunction in the nervous system. The devices and methods may additionally provide relief of other abnormal neurologic activity, such as, for example, discomfort associated with conditions known as a “charlie horse” or related muscles cramps, or rigidity of muscles experienced by victims of a stroke.

While the exact physiological mechanisms acting to provide relief of symptoms using these therapeutic devices and methods of treatment are not completely understood, it is thought that the light tactile stimulation provides a form of peripheral nerve distraction, thus interrupting, masking, or otherwise diverting abnormal muscle impulses and/or sensations. Indeed, the effectiveness of the devices and methods of treatment were unexpected and surprising. The common features of the alternative constructions of the therapeutic devices is an ability to tend to hold a device in a selected location on a body part and to apply light tactile stimulation to an affected region of the body part to treat the abnormal neurologic activity. Typical treatment areas or body parts to be treated would include at least the arms, legs, and knees, although it is contemplated that successful treatment of body parts also may include other areas of the body. In use, it is believed that such devices and methods of treatment provide significant benefits with respect to costs and risks relative to the prior art.

In a first aspect of the disclosure, a therapeutic device for reducing the incidence and severity of symptoms related to abnormal neurologic activity within a body part is disclosed. The therapeutic device includes at least one holding implement configured to position the therapeutic device on the body part and at least one therapeutic implement connected to the holding implement and configured to provide light tactile stimulation to a region of the body part that is in the vicinity of the holding implement.

In another aspect of the disclosure, a method for reducing the incidence and severity of symptoms related to abnormal neurologic activity within a body part is disclosed. The method includes applying to the body part a therapeutic device having at least one therapeutic implement connected to at least one holding implement. Applying the therapeutic device to the body part also includes locating the at least one holding implement in a desired position on the body part and locating the at least one therapeutic implement adjacent to a region of the body part that is in the vicinity of the holding implement, with the at least one therapeutic implement configured to provide light tactile stimulation to the body part.

Consistent with the present disclosure, a holding implement is used to locate the therapeutic device in a desired position on a body part. The holding implement is intended to hold the device in a particular selected position. The holding implement may be in the form of a band. The band may be constructed as a continuous loop to encircle a body part. The band also may be constructed as a strap having two ends and which can be wrapped around a body part.

Given that the band can be constructed with elastic or inelastic materials or fabrics, the band may be self adjusting, so that it will fit a variety of body part circumferences and not restrict circulation within the body part with excessive pressure. One advantage of having at least one holding implement be elastic is that it allows the circumference of the body part to change slightly, such as when a calf or arm muscle is flexed or relaxed, or encounters swelling, without causing tightness or looseness in the fit of the therapeutic device. It is contemplated that the holding implement alternatively may be manually adjustable.

It also is contemplated that the holding implement could have other configurations than a band and could rely on different means to tend to hold the therapeutic device in place. For instance, the holding implement could be of a construction that utilizes a patch or strip having a temporary adhesive, such as is used with some bandages, or other suitable means.

The therapeutic implement is configured to provide light tactile stimulation to the affected body part. The therapeutic implement is to fit the body part in a range from touching the body part (or the skin) with no pressure, where the therapeutic implement fits snugly enough to hold it against the surface of the body part without applying pressure, to a light and loose fit, where the therapeutic implement is so loose that it is free to slouch, or simply hangs and makes less predictable contact with the body part.

As in some examples disclosed herein, the therapeutic implement also may be in the form of a band, whether configured as a continuous loop or a strap having connectable opposed ends. Such bands may be constructed with elastic or inelastic materials or fabrics. Further alternative constructions to affect the application of light tactile stimulation may include, for example, vertically or horizontally oriented straps, tassles, strands, strings of beads, or other suitable constructions.

The therapeutic implement is configured to provide light tactile stimulation and when in the form of a strap, may be adjustable in length. Thus, the strap may be provided with a hook and loop fastener, enabling easy adjustment of the strap fit. A person using the device would thereby be able to readily adjust the lightness of tactile stimulation within a range from touching the body part with essentially no compression to a light and loose fit. This provides the user the opportunity to discover the tactile stimulation that proves to be most effective for reducing symptoms in his or her particular body part at a particular time. Ideally, once a preferred position is determined, the user may remove the device from the body part when necessary and simply reapply it, locating the device in the previous treatment position at a later time, without having to readjust the holding or therapeutic implements. However, it may be that the tactile stimulation provided by touching the body part with no pressure is effective at one time, while a light and loose fit is needed for the greatest sense of relief at another time, or vice versa.

In another aspect of the disclosure, a therapeutic device may have a series of adjustable therapeutic implements, so that the device can be easily adjusted to provide light tactile stimulation on a body part having various sizes and shapes. For example, one therapeutic implement may be a strap that may be adjusted to provide a light and loose fit in one region of a calf while another may be a strap that may be adjusted to provide a light and loose fit in another region of the leg or calf having a different circumference. The series of straps also can be easily adjusted to provide light tactile stimulation of one level on one region of a body part and to provide light tactile stimulation of another level at another region of the body part. For example, one strap may be adjusted to provide a light and loose fit in one region of the calf and another strap may be adjusted to touch the body part more completely with no pressure in another region of the leg or calf, regardless of whether the regions have the same or different circumferences.

As previously mentioned, a therapeutic implement may be provided in the form of a band that is constructed as an adjustable strap. It will be appreciated that such adjustability may be provided by using an aforementioned hook and loop fastener, or by suitable alternatives, such as a buckle, a series of snaps, a button and a series of button holes, or any other appropriate constructions.

It is contemplated that the therapeutic devices will have at least one therapeutic implement connected to at least one holding implement. The respective implements may be connected in various suitable ways for ease of use in applying the device to a body part. For instance, the therapeutic and holding implements may be connected directly or to a further common central portion. The central portion also may permit further adjustment of implements with respect to each other. Alternatively, the implements may be constructed from a unitary piece with an integral central portion. With the treatment methods disclosed, if a therapeutic device includes a central portion, it may be more effective to orient the therapeutic device such that the common central portion is not located on the muscle of the body part to be treated. For example, if the treatment region includes the calf muscle of the lower leg, it is believed, though not required, that positioning of the central region of the therapeutic device over the opposed shin will yield the most effective relief.

In a further aspect of the disclosure, a method is presented for reducing the incidence and severity of symptoms related to abnormal neurologic activity by applying a therapeutic device that applies light tactile stimulation to the affected body part and provides warmth in the region to be treated. Thus, the material utilized in constructing the therapeutic implements may be selected to provide warmth to the affected body part, in addition to providing light tactile stimulation. For example, neoprene fabric may be laminated to hook compatible loop fabric to provide a degree of warmth to a body part in addition to providing light tactile stimulation. While the exact physiological mechanisms acting to provide relief of symptoms are not completely understood, it is thought that warmth in conjunction with light tactile stimulation may interrupt, mask, or otherwise provide peripheral nerve distraction to divert abnormal muscle impulses and/or sensations.

Excessive warmth may cause discomfort or moisture accumulation on the skin of the body part. Accordingly, the material utilized in the therapeutic implement may be Breathoprene®, which is a neoprene having a series of apertures to provide ventilation and/or to moderate the warming effect. Alternatively, the surface of the therapeutic implements contacting the body part may be lined with a fabric known to wick away moisture, such as CoolMax® or polypropylene. It is understood that the material selected for the therapeutic implements, the addition of apertures through the material, and the selection of fabrics or materials lining the therapeutic implements may be determined to provide a desired level of comfort and therapeutic effect.

It also has been observed through practice of the subject matter of this disclosure that the therapeutic effect of providing light tactile stimulation to an affected body part may be obtainable even when the stimulation is provided to the surface of a bandage or wrap that has been placed on the body part. For example, a patient wearing a bandage wrap for the treatment of open sores on a leg may obtain relief from RLS symptoms by wearing a therapeutic device consistent with this disclosure in a position over the bandage wrap.

### BRIEF DESCRIPTION OF THE DRAWINGS

FIG. 1 is a perspective view of a first example of a therapeutic device.

FIG. 2 is a perspective view of a second example of a therapeutic device positioned on a lower leg.

FIG. 3 is a perspective view of the second example therapeutic device of FIG. 2, in an open and flattened position.

FIG. 4 is a perspective view of a third example therapeutic device, in an open and flattened position.

FIG. 5 is a perspective view of a fourth example of a therapeutic device.

FIG. 6 is a perspective view of a fifth example of a therapeutic device.

### DETAILED DESCRIPTION

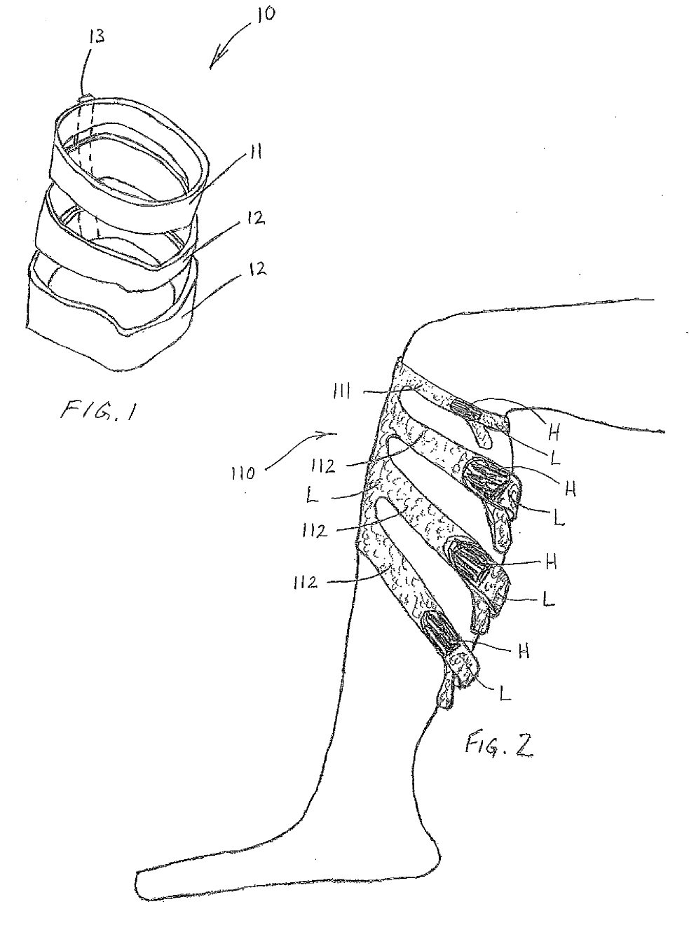

Referring generally to FIGS. 1-6 it will be appreciated that therapeutic devices used in methods of treatment to reduce the incidence and severity of symptoms related to abnormal neurologic activity may be embodied within numerous configurations.

Referring to a first example shown in FIG. 1, a therapeutic device 10 is constructed with one holding implement 11 and a plurality of therapeutic implements 12. The implements 11, 12 are arranged in a relationship to enable the device to encompass a body part, such as a leg. The holding implement 11 allows the device 10 to generally maintain a selected position on the desired body part, without applying significant pressure. The therapeutic implements 12 provide light tactile stimulation to the body part. In this example, the holding implement 11 preferably is constructed from an elastic material that is sewn into a circular band or continuous loop. In addition, the therapeutic implements 12 preferably are constructed from elastic fabric sewn into a circular band or continuous loop sized to fit the body part in a range from snug with no pressure to a light and loose fit. In this example, the holding implement 11 and therapeutic implements 12 are joined to a common central portion 13.

Utilizing the example therapeutic device 10 in a method for reducing the incidence and severity of symptoms related to abnormal neurologic activity within a body part would include applying the therapeutic device 10 to the body part. The therapeutic device 10 further includes at least one holding implement 11 to tend to hold the device in a selected position on the body part, and at least one therapeutic implement 12 to apply light tactile stimulation to the body part in a range from touching the skin without pressure to a light and loose fit. The method of treatment may further include a step of locating the holding implement on the body part, such as by encircling a leg with the at least one holding implement 11. This may be accomplished by sliding the at least one holding implement 11 into a position to hold the therapeutic device 10 on the body part. The method further would include the step of locating at least one therapeutic implement 12 adjacent a region of the body part that is in the vicinity of the holding implement 11. In this example, this may be accomplished by encircling the body part with the at least one therapeutic implement 12 to apply light tactile stimulation to the body part in a range from touching the skin without pressure to a light and loose fit.

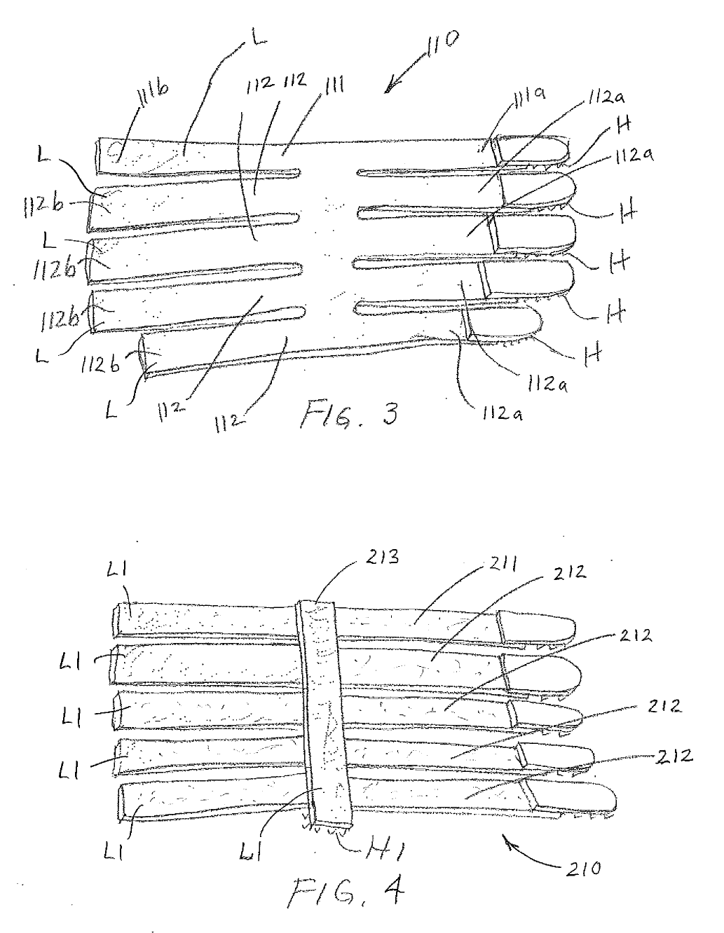

A second example of a therapeutic device is shown in FIGS. 2 and 3. The device is positioned on a lower leg in FIG. 2 and in an open and flattened position in FIG. 3. In the second example, a therapeutic device 110 is constructed with one holding implement 111 and a plurality of therapeutic implements 112 arranged in a relationship to enable the device to encompass a body part, such as a leg. In this example, the main portion of the holding implement 111 and the therapeutic implements 112 are constructed as bands, in the form of straps having two ends. The implements 111, 112 also are constructed from a unitary piece, which may include one or more layers of fabric.

In the example therapeutic device 110, the fabric has a loop surface L suitable for fastening to a hook portion H of a hook and loop fastener on at least one side of the device 110. At a first end 111a of the holding implement 111, and at a first end 112a of each therapeutic implement 112, a hook portion H is applied. The hook portion H is affixed to and extends from the loop surface L of the strap ends 111a, 112a. In use, the holding implement 111 encircles the body part, as seen just above the calve muscle in FIG. 2, and is adjusted to a circumference that tends to hold the device in a selected position on the body part. The adjustment is made by use of a hook portion H on the first end 111a being fastened to a loop surface L on a second end 111b of strap 111, which allows the strap to retain a desired size. In this example, the strap 111 is adjusted so as to be stopped from sliding downward and over the calve portion which has a greater circumference than the portion of the leg below the calve muscle.

In turn, each therapeutic implement 112 encircles the body part and is adjusted to a circumference that provides a fit ranging from snug with no pressure to a light and loose fit on the limb. For each therapeutic implement 112, a hook portion H on the first end 112a is fastened to a loop surface L on the second end 112b to allow the respective strap to retain a desired size by which the device 110 will provide light tactile stimulation. It will be appreciated that, as illustrated, a therapeutic device may have a length of for instance, 8″ when flattened, but when hanging from a body part, such as a lower leg, it may droop to the point of being at least a few inches longer in length.

In the second example shown in FIGS. 2 and 3, the holding and therapeutic implements are preferably made from a piece of neoprene or Breathoprene® fabric having one surface bonded to a lightweight loop fabric suitable for hook and loop fastening and forming a unitary piece. The other side of the neoprene or other suitable material may be plain, although it preferably will be bonded with a fabric suitable for contact with the skin, such as nylon or polypropylene, and preferably will retain the elastic nature of the materials. When applying the device to a body part, such as a leg, the holding implement 11 may be located above the largest circumference of the calf and secured to tend to hold the therapeutic device 110 in place, such as by preventing it from sliding downward on the leg. The remaining straps include therapeutic implements 112 for applying light tactile stimulation to the surface of the affected body part. Each of the therapeutic implements 112 is wrapped around the body part, but the straps are adjusted to fit in a range from snug with no pressure to a light and loose fit when the hook portion H is engaged with the loop surface L. For example, the strap end 112a is wrapped around the body part and the hook portion H is engaged with the loop surface L on the strap end 112b.

The second example therapeutic device 110 may be used in a method for reducing the incidence and severity of symptoms related to abnormal neurologic activity within a body part. The method of use of therapeutic device 10 would include applying the therapeutic device 110 to the body part. The therapeutic device 110 further includes at least one holding implement 111 to tend to hold the device in a selected position on the body part, and at least one therapeutic implement 112 to apply light tactile stimulation to the body part in a range from touching the body part without pressure to a light and loose fit. The method of treatment may further include a step of locating the at least one holding implement 111 in a desired position on the body part. The method further would include the step of locating the at least one therapeutic implement 112 on the body part to apply light tactile stimulation to the body part in a range from touching the body part without pressure to a light and loose fit. The method also may include adjusting the implements 111, 112, as well as the at least one therapeutic implement 112 applying warmth to the body part in a selected region to be treated by the therapeutic device.

FIG. 4 presents another example therapeutic device 210. The therapeutic device 210 is constructed with a holding implement 211 and therapeutic implements 212, which may be connected by a central portion 213. The holding implement 211 and therapeutic implements 212 are constructed in the form of bands that are configured as straps being adjustable in length. The holding and therapeutic implements 211, 212 may be connected to the central portion 213 by sewing, bonding, use of releasable fasteners or other suitable fastening means. For example, the central portion 213 may be constructed of a piece of hook material H1 that is fastened to a hook compatible loop surface L1 on the implements 211, 212. One or more holding implements 211 and one or more therapeutic implements 212 may be connected to the central portion 213 as desired to hold the therapeutic device in place and provide light tactile stimulation to the desired body part.

With the example therapeutic device 210 shown in FIG. 4, the holding and therapeutic implements 211, 212 need not all be connected to the central portion 213 in the same manner. For instance, the holding implement 211 may be sewn to the central portion 213, while each therapeutic implement 212 may be selectively connected to the central portion 213 by use of hook and loop fastening means. This would permit the therapeutic implements 212 to be repositioned relative to the holding implement 211 and/or relative to each other and used in various quantities, such as one or more therapeutic implements 212, as desired. In addition, the central portion 213 may include suitable hook portion H1 on a first side and a complementary loop surface L1 on the opposite side to permit the central portion 213, when constructed as a strap, to be connected to itself, such as when fewer therapeutic implements 212 are used and the user wants to avoid having the extra length of the central portion 213 hang freely.

The third example therapeutic device 210 may be used in a method for reducing the incidence and severity of symptoms related to abnormal neurologic activity within a body part, in a somewhat similar manner to the second example therapeutic device 110. However, as noted above, the therapeutic device 210 may provide increased adjustability with respect to the manner in which the therapeutic implements 212 are connected to the central portion 213. Thus, the method may include further steps of adjusting the number of therapeutic implements 212 and the positions of the respective therapeutic implements 212 relative to each other and to the holding implement 211.

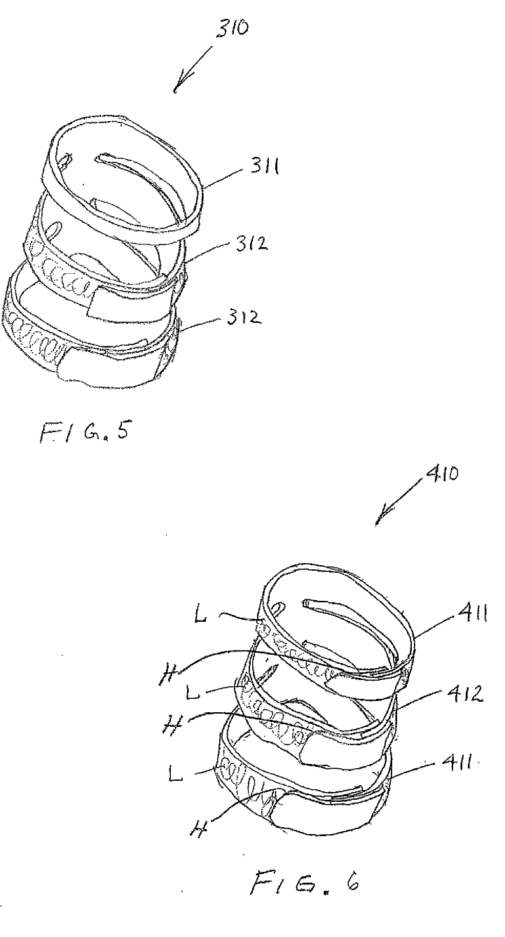

in a further example therapeutic device shown in FIG. 5, a therapeutic device 310 is held in place with one holding implement 311, in the form of a band having a continuous loop, and further includes one or more therapeutic implements 312, constructed as bands in the form of straps which can be adjusted to provide light tactile stimulation to the affected body part. In this example, the holding implement 311 likely is constructed of an elastic material, to accommodate variations in the circumference of a body part. Due to the unitary piece construction of therapeutic device 310, the therapeutic implements 312 also would be elastic, which would provide for greater adjustability.

The method of using the therapeutic device 310 of the fourth example is somewhat of a hybrid between the method of use of the first example shown in FIG. 1 and the second example shown in FIGS. 2 and 3. This is because the holding implement 311 is elastic but not otherwise adjustable, while the at least one therapeutic implement 312 are more freely adjustable in length. Thus, the method of treatment may further include a step of encircling the body part with the at least one holding implement 311 and sliding the at least one holding implement 311 into a position to tend to hold the therapeutic device on the body part. The method further would include the step of encircling the body part with the at least one therapeutic implement 312 to apply light tactile stimulation to the body part in a range from touching the body part without pressure to a light and loose fit. In this example, the at least one therapeutic implement 312 is adjustable, which permits further adjustments between applications of the device, if desired.

FIG. 6 presents another example therapeutic device 410. With this construction, the therapeutic device 410 tends to be held in place with two holding implements 411 constructed as bands in the form of adjustable straps, and being located at the top and bottom of the device. The device 410 further includes one or more therapeutic implements 412 providing light tactile stimulation to the affected body part. As illustrated by these further examples, it is contemplated that many combinations of holding implements arid/or therapeutic implements, whether in the form of continuous bands, straps or other suitable constructions as previously mentioned, would be suitable to provide the light tactile stimulation suitable for the disclosed treatment.

The fifth example therapeutic device 410 may be used in a method for reducing the incidence and severity of symptoms related to abnormal neurologic activity within a body part, in a somewhat similar manner to the second example therapeutic device 110. However, as noted above, the therapeutic device 410 includes holding implements 411 in upper and lower positions on the device. Thus, the method may include applying at least one holding implement 411 in an upper position to hold the device in a selected position on the body part, and applying at least one holding implement 411 in a lower position to tend to hold the device 410 in the selected position on the body part. The method may further include the step of locating the at least one therapeutic implement 412 adjacent to a region of the body part that is in the vicinity of the holding implement 411, with the therapeutic implement 412 configured to apply light tactile stimulation to the body part. The stimulation would preferably be in a range from touching the body part without pressure to a light and loose fit.

Turning to more general construction characteristics of the examples shown herein, the holding implements I 1, 311 and holding implements 111, 211, 411 are sized in a length and width suitable for tending to hold the therapeutic device in a selected position on the intended body part. For example, a width of 1″ has been successfully utilized on devices used on arms and legs, although widths wider or narrower may be used. The length of holding implements depends on the configuration of the holding implement and the way in which it will be applied to the body part, such as by encircling with a continuous loop, encircling with a strap, adhesion to the body part or other suitable alternatives.

In the examples shown, the therapeutic implements 12 and therapeutic implements 112, 212, 312, 412 are similarly sized in a length suitable for encircling the desired body part. It is understood that the device may be constructed in more than one size, and that each size would be capable of fitting body part circumferences within a specific range. For example, a small size may be designed to fit body part circumferences ranging from 6″ to 12″, a medium size may be designed to fit circumferences ranging from 9″ to 16″, and so forth. The therapeutic implements may be provided with each being the same length or may vary in length, in accordance with the general contour of a selected body part. For example, the therapeutic implements 112, 212, 312, 412 of a device designed for a lower leg may be longer in the region of the maximum circumference of the calf and shorter in the region closer to the ankle, or they all may be the same length. In instances where vertically hanging therapeutic implements are used, they also may be of similar or different lengths.

In application, excess length of a holding implement that is constructed as a band and in the form of a strap having two ends may be trimmed or left on the device, with the holding implement overlapping itself to the degree that the length of the implement exceeds the circumference of the body part to be treated. In the same way, excess length of each similarly constructed therapeutic implement that is provided may be trimmed or left on the device, with the therapeutic implement overlapping itself to the degree that the length of the implement exceeds the circumference of the body part. While any excess length of a holding and/or therapeutic implement may be removed, the excess length of either alternatively may be permitted to hang and act to provide additional light tactile stimulation to the body part, thereby adding to the therapeutic effect. It also is anticipated that one or more of the therapeutic implements may be trimmed or otherwise removed from the device if the length of the entire therapeutic device exceeds the desired length for treating the intended body part.

The therapeutic implements 12 and therapeutic implements 112, 212, 312, 412 are to be sized in a width suitable for providing light tactile stimulation to the body part at a level that provides therapeutic reduction in the incidence and severity of symptoms related to abnormal neurologic activity. The most effective width of a therapeutic implement in the form of the examples shown herein may vary depending upon the material selected and the circumference of the body part that the implement is intended to encircle. For example, if the fabric of the therapeutic implement is not very stiff and the implement is fashioned in a narrow width, then the implement may tend to roll upon itself and present a reduced contact surface to the body part, thereby providing a light tactile touch that is different from that provided by a fabric implement that lays flat against the body part. If this occurs, the therapeutic implement may be initially adjusted to provide light tactile stimulation that is judged to be optimal by the wearer, only to have the tactile stimulation provided change as the implement rolls upon itself. In this case, the minimum width of a therapeutic implement may be wider than is required if the material were to have greater stiffness, such as with neoprene that is laminated to loop fabric.

The maximum desired width of an example therapeutic implement 12 or therapeutic implement 112, 212, 312, 412 is similarly limited according to the stiffness and conformability of the material utilized in the therapeutic implement construction. For example, a single 4″ wide implement constructed of a material having a high stiffness will have more limited surface contact with a contoured body part than two 2″ wide implements constructed of the same material. This is due to the fact that the 4″ wide implement may contact a relatively small surface area and bridge surrounding areas having a smaller circumference. In this example, a 2″ wide implement may similarly contact a relatively small surface area and bridge surrounding areas, but a second 2″ wide implement will contact a portion of the area that would have been bridged by the single 4″ wide implement. In this way, two 2″ wide implements provide light tactile stimulation to a greater portion of the body part than a single 4″ wide implement.

In an example of a therapeutic device, such as shown in FIGS. 2 and 3, the therapeutic implements 112 may be constructed of 2 mm neoprene laminated to hook compatible loop fabric and having widths ranging from 1″ to 2″ wide and sized in lengths suitable for an arm or lower leg. Such a device has successfully reduced the incidence and severity of symptoms related to abnormal neurologic activity. It is understood that therapeutic implements having widths wider than or narrower than this range, or employing other constructions, such as beaded strings, strands, tassles or other structures, also may be effective, depending on the specific user and affected body part.

It will be appreciated that a therapeutic device in accordance with this disclosure may be provided in various configurations and still provide at least one holding implement and at least one therapeutic implement that provides light tactile stimulation or light tactile stimulation and warmth in a range that reduces the incidence and severity of symptoms related to abnormal neurologic activity. Any variety of suitable materials of construction, configurations, shapes, and sizes for the components and methods of connecting the components may be utilized to meet the particular needs and requirements of the person to be treated. It will be apparent to those of ordinary skill in the art that various therapeutic devices may be constructed and methods of treatment employed without departing from the scope or spirit of the present disclosure. Thus, although certain example devices and methods have been described herein, the scope of coverage of this patent is not limited thereto. On the contrary, this patent covers all devices and methods fairly falling within the scope of the appended claims either literally or under the doctrine of equivalents.

What is claimed is:

1. A therapeutic device for reducing the incidence and severity of symptoms related to abnormal neurologic activity within a body part comprising:
at least one holding implement configured to position the therapeutic device on the body part; and
at least one therapeutic implement connected to the holding implement and configured to provide light tactile stimulation to a region of the body part that is in the vicinity of the holding implement.

2. The therapeutic device defined in claim 1, wherein the at least one holding implement includes at least one band.

3. The therapeutic device defined in claim 2, wherein the at least one band is a continuous band configured to encircle the body part.

4. The therapeutic device defined in claim 3, wherein the at least one band is constructed from an elastic fabric.

5. The therapeutic device defined in claim 2, wherein the at least one band is formed by a strap.

6. The therapeutic device defined in claim 5, wherein the strap is adjustable to hold the device on the body part.

7. The therapeutic device defined in claim 1, wherein the at least one therapeutic implement includes at least one band.

8. The therapeutic device defined in claim 7, wherein the at least one band is configured to have a range of fit to the body part from being snug without pressure to a light and loose fit.

9. The therapeutic device defined in claim 7, wherein the at least one band is constructed from an elastic fabric.

10. The therapeutic device defined in claim 7, wherein the at least one therapeutic band is formed by a strap.

11. The therapeutic device defined in claim 10, wherein the strap is adjustable to have a range of fit to the body part from being snug without pressure to a light and loose fit.

12. The therapeutic device defined in claim 1, wherein the at least one therapeutic implement is constructed to be able to provide warmth to the body part.

13. The therapeutic device defined in claim 1, wherein the at least one holding implement and at least one therapeutic implement are constructed as a unitary piece.

14. The therapeutic device defined in claim 1, wherein the at least one holding implement and at least one therapeutic implement are connected to a central portion.

15. The therapeutic device defined in claim 14, wherein the at least one holding implement is affixed to the central portion and the at least one therapeutic implement is releasably connectable to the central portion.

16. A method for reducing the incidence and severity of symptoms related to abnormal neurologic activity within a body part comprising:
applying to the body part a therapeutic device having at least one therapeutic implement connected to at least one holding implement, wherein applying the therapeutic device to the body part further comprises:
locating the at least one holding implement in a desired position on the body part; and
locating the at least one therapeutic implement adjacent to a region of the body part that is in the vicinity of the holding implement, with the at least one therapeutic implement configured to provide light tactile stimulation to the body part.

17. The method defined in claim 16, wherein applying to the body part a therapeutic device further includes the step of encircling the body part with the at least one holding implement.

18. The method defined in claim 16, wherein applying to the body part a therapeutic device further includes the step of encircling the body part with the at least one therapeutic implement.

19. The method defined in claim 16, wherein the at least one therapeutic implement is configured to have a range of fit to the body part from being snug without pressure to a light and loose fit.

20. The method defined in claim 16, wherein applying to the body part a therapeutic device further includes the step of adjusting the therapeutic implement to have a fit in a range from touching the body part without pressure to a light and loose fit.

21. The method defined in claim 16, wherein the at least one therapeutic implement further provides warmth to the body part in a selected region to be treated by the therapeutic device.
